(19) United States
(12) Patent Application Publication
(10) Pub. No.: US 2025/0049507 A1
(43) Pub. Date: Feb. 13, 2025

(54) ENHANCED USER INTERFACE AND CROSSTALK ANALYSIS FOR VASCULAR INDEX MEASUREMENT

(71) Applicant: Cathworks Ltd., Kfar Saba (IL)

(72) Inventors: Ifat Lavi, Moshav Mishmeret (IL); Moran Shalhon Livne, Rosh Haayin (IL)

(21) Appl. No.: 18/821,934

(22) Filed: Aug. 30, 2024

Related U.S. Application Data
(63) Continuation of application No. PCT/IB2024/057718, filed on Aug. 9, 2024.
(60) Provisional application No. 63/518,529, filed on Aug. 9, 2023.

(51) Int. Cl. A61B 34/10; G16H 50/30; G16H 50/50

(52) U.S. Cl. CPC A61B 34/10; A61B 34/25; G16H 50/30; G16H 50/50; A61B 2034/105

(57) ABSTRACT

Systems and methods for enhanced user interface and crosstalk analysis for cardiac index determination. An example method includes accessing a cardiac model of a portion of a patient's heart, the portion including one or more vessels of the patient's heart, and the cardiac model indicating a plurality of lesions along a length of at least one of the vessels; obtaining, based on the cardiac model for the lesions, respective positions along the length for which the lesions are associated with index drops, wherein the index drops are with respect to an index indicative of vascular function; and causing presentation of a user interface, wherein the user interface: presents a graph mapping the length to the index indicative of vascular function, presents individual toggles enabling nulling of individual lesions, and updates the graph in response to received user input to one or more of the toggles, wherein the user input nulls effects of one or more lesions.

### CROSS-REFERENCE TO RELATED APPLICATIONS

This application claims priority to, and is a continuation of, International Patent App. No. PCT/IB2024/057718 titled “ENHANCED USER INTERFACE AND CROSSTALK ANALYSIS FOR VASCULAR INDEX MEASUREMENT” and filed on Aug. 9, 2024, which claims priority to U.S. Prov. Patent App. No. 63/518,529 titled “ENHANCED USER INTERFACE AND CROSSTALK ANALYSIS FOR VASCULAR INDEX MEASUREMENT” and which was filed on Aug. 9, 2023. The entire disclosure of each of the above-identified applications is hereby incorporated herein by reference in its entirety.

### BACKGROUND

Cardiovascular disease (CVD) is a leading cause of morbidity and mortality, with an estimated 244.1 million people worldwide with CVD, particularly due to the subsect of CVD, coronary artery disease (CAD). CAD may involve a prolonged asymptomatic developmental phase, with clinical manifestations often resulting in angina pectoris, acute myocardial infarction (MI), or cardiac death. The underlying mechanism that may cause CAD involves atherosclerosis of the coronary arteries. Atherosclerosis is a plaque buildup that narrows the coronary arteries and decreases blood flow to the heart, resulting in ischemia or coronary stenosis.

Revascularization may be the preferred therapy for patients with moderate to severe ischemia or stenosis, resulting in significant improvements for the patient due. Revascularization strategies include many techniques such as open-heart surgery, coronary artery bypass grafting (CABG), and percutaneous coronary intervention (PCI) methods such as balloon angioplasty, bare-meta stents (BMS), and first- and second-generation drug-eluting stents (DES). The severity of CAD can be assessed through vascular computer models.

### SUMMARY

The disclosure generally contemplates systems and methods for determining the effectiveness of percutaneous coronary intervention (PCI) using non-invasive techniques based on cross-talk analysis.

In some aspects, the techniques described herein relate to a method implemented by a system of one or more processors, the method including: accessing a cardiac model of a portion of a patient's heart, the portion including one or more vessels of the patient's heart, and the cardiac model indicating a plurality of lesions along a length of at least one of the vessels; obtaining, based on the cardiac model for the lesions, respective positions along the length for which the lesions are associated with index drops, wherein the index drops are with respect to an index indicative of vascular function; and causing presentation of a user interface, wherein the user interface: presents a graph mapping individual positions along the length of the vessel and values of the index indicative of vascular function, presents individual user interface elements enabling nulling of individual lesions, and updates the graph in response to received user input to one or more of the user interface elements, wherein the user input nulls effects of one or more lesions.

In some aspects, the techniques described herein relate to a method, wherein the user interface elements are toggles.

In some aspects, the techniques described herein relate to a method, wherein the plurality of lesions includes at least one user-identified lesion.

In some aspects, the techniques described herein relate to a method, wherein the plurality of lesions further includes at least one automatically-identified lesion that meets an index drop threshold.

In some aspects, the techniques described herein relate to a method, wherein the cardiac model is a three-dimensional model generated based on angiographic images.

In some aspects, the techniques described herein relate to a method, wherein the index indicative of vascular function is a fractional flow reserve value.

In some aspects, the techniques described herein relate to a method, wherein the graph is a line.

In some aspects, the techniques described herein relate to a method, wherein the user input triggers a determination of index drops associated with remaining lesions.

In some aspects, the techniques described herein relate to a method, wherein nulling a lesion cause updating of diameters of the vessel which are associated with the lesion, and wherein the updated diameters are updated to cause index drops less than a threshold.

In some aspects, the techniques described herein relate to a method, wherein the user interface updates to include an updated index indicative of vascular function.

In some aspects, the techniques described herein relate to a system including one or more processors and non-transitory computer storage media storing instructions that when executed by the one or more processors, cause the one or more processors to perform the method.

In some aspects, the techniques described herein relate to non-transitory computer storage media storing instructions that when executed by a system.

In some aspects, the techniques described herein relate to a method implemented by a system of one or more processors, the method including: accessing a cardiac model of a portion of a patient's heart, the portion including one or more vessels of the patient's heart, and the cardiac model indicating a plurality of lesions along a length of at least one of the vessels; obtaining, based on the cardiac model for the lesions, respective positions along the length for which the lesions are associated with index drops, wherein the index drops are with respect to an index indicative of vascular function for the vessels; and causing presentation of a user interface, wherein the user interface: presents individual user interface elements enabling nulling of individual lesions, and updates the index in response to received user input to one or more of the user interface elements, wherein the user input nulls effects of one or more lesions.

In some aspects, the techniques described herein relate to a method, wherein the user interface elements are toggles.

In some aspects, the techniques described herein relate to a system including one or more processors and non-transitory computer storage media storing instructions that when executed by the one or more processors, cause the one or more processors to perform the method.

In some aspects, the techniques described herein relate to non-transitory computer storage media storing instructions that when executed by a system.

In some aspects, the techniques described herein relate to a method implemented by a system including one or more processors, the method including: accessing a cardiac model of a portion of a patient's heart, the portion including one or more vessels of the patient's heart, and the cardiac model indicating a plurality of lesions along a length of at least one of the vessels; obtaining, based on the cardiac model for the lesions, respective positions along the length for which the lesions are associated with index drops, wherein the index drops are with respect to an index indicative of vascular function for the vessels; and based on information indicating that a particular lesion of the plurality of lesions is to be nulled, updating the index indicative of vascular function to null effects of the particular lesion.

In some aspects, the techniques described herein relate to a method, wherein updating the index is based on addressing crosstalk associated with remaining lesions.

In some aspects, the techniques described herein relate to a method, wherein the particular lesion is downstream from one or more remaining lesions.

In some aspects, the techniques described herein relate to a method, wherein the particular lesion is between adjacent of the lesions.

In some aspects, the techniques described herein relate to a system including one or more processors and non-transitory computer storage media storing instructions that when executed by the one or more processors, cause the one or more processors to perform the method.

In some aspects, the techniques described herein relate to non-transitory computer storage media storing instructions that when executed by a system.

The systems, methods, techniques, modules, and devices of this disclosure each have several innovative aspects, no single one of which is solely responsible for the desirable attributes disclosed herein.

### BRIEF DESCRIPTION OF THE DRAWINGS

FIG. 1 is a block diagram of an example lesion impact system generating an adjustable lesion graph of a targeted vessel.

FIG. 2A illustrates an example adjustable lesion graph with three lesions.

FIG. 2B illustrates the example adjustable lesion graph with a first lesion toggled to null the lesion.

FIG. 2C illustrates the example adjustable lesion graph with a third lesion toggled to null the lesion.

FIG. 2D illustrates the example adjustable lesion graph with the first and the third lesions toggled to null the lesions.

FIG. 3 is a flowchart of an example process for generating a user interface which is responsive to user input associated with toggling lesions included in an adjustable lesion graph.

FIG. 4 is a user interface illustrating a vascular assessment that includes an adjustable lesion graph.

FIG. 5 is a user interface illustrating another example of a vascular assessment that includes an adjustable lesion graph.

FIG. 6 is a user interface illustrating a comparison between the two example adjustable lesion graphs.

### DETAILED DESCRIPTION

### Overview

This specification describes techniques to more accurately, and non-invasively, characterize a patient's cardiac health options. As will be described, cardiac images (e.g., angiographic images) of a patient may be obtained. A system or user device may analyze the images to determine a set of lesions that may be negatively impacting the patient's health. A lesion, as may be appreciated, may be associated with a narrowing or constriction of a cardiac vessel. As known by those skilled in the art, due to safety, it may be impractical to revascularize all lesions of the determined vessel (e.g., via percutaneous coronary intervention). The disclosed technology enables medical professionals to simulate the effects of nulling particular lesions of a vessel and determine which lesion or lesions have the highest potential impact on vessel stenosis. Nulling a lesion, as will be described, may simulate removal of the lesion or causing the cardiac vessel in a location of the lesion to be adjusted in size. For example, diameters of the location may be adjusted to be normal (e.g., healthy) or similar to other locations of the vessel which are not associated with stenosis.

For example, and as described herein, an adjustable lesion graph (e.g., graph 130) may be generated which allows the medical professional to null the effects of lesions with respect to a score or index of cardiac health (collectively referred to herein as an index or index indicative of vascular function). An example index may include a fractional flow reserve value (FFR value). In this example, the adjustable lesion graph may depict 2, 3, 4, 5, and so on, lesions. Each of these lesions may be associated with a reduction in the index indicative of vascular function (referred to herein as an index drop).

A medical professional can null some, or all, of the lesions via user interface elements (e.g., toggles) included in a user interface. In some embodiments, the graph may include all lesions as causing index drops and the medical professional may null a subset of the lesions. When a lesion is nulled, the user interface may then update to include an updated score or index. As an example, the medical professional may null a particular lesion, such that its effects are eliminated, by interacting with a toggle included in the user interface. For this example, the index may update in substantially real-time. As an example, the graph may update such that the indices included in the graph (e.g., the index drops) are adjusted based on nulling the particular lesion. As described herein, toggling a lesion on, or toggling on a lesion, may indicate interaction with a user interface element which causes, in some embodiments, nulling of the lesion. Similarly, a lesion which has been toggled on is referred to herein as a nulled lesion.

Nulling a single lesion may represent the narrowing or constricting of a vessel being eliminated or substantially reduced. For example, nulling may represent the vessel's diameter being increased such that blood flow is not impeded (e.g., not impeded, not impeded more than normal, and so on). In this way, the nulled single lesion will not have an index drop or will not have an index drop greater than a threshold. Thus, when nulling a single lesion, a system or user device may determine an updated index indicative of vascular function based on a simulation of the lesion area as a healthy vessel.

However, nulling the single lesion may impact the index determination in complex ways which, at present, software-based techniques do not accurately capture. For example, the single lesion may have one or more lesions upstream and/or one or more lesions downstream. In this example, crosstalk effects between the lesions may cause non-linear adjustments to the index. Thus, simply simulating an index drop associated with the single lesion from an overall index may be inaccurate. The disclosed technology therefore ensures that such crosstalk effects are considered such that the overall index is accurately determined.

Additional disclosure related to determining an index indicative of vascular function and adjusting the index based on nulling lesions, and so on, are described in more detail in U.S. Pat. Nos. 10,595,807, 11,138,733, and 9,814,433 which are hereby incorporated herein by reference in their entireties.

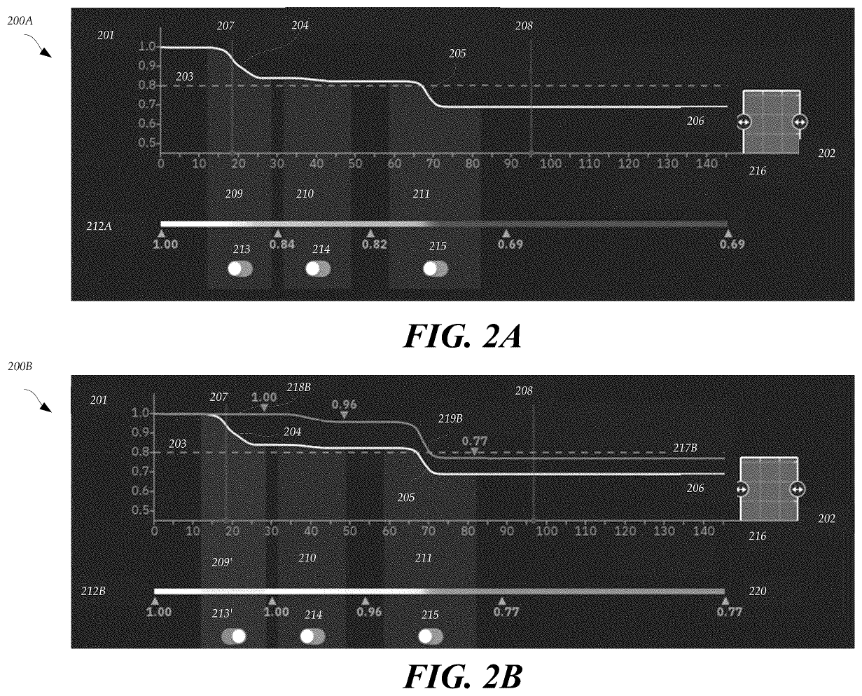

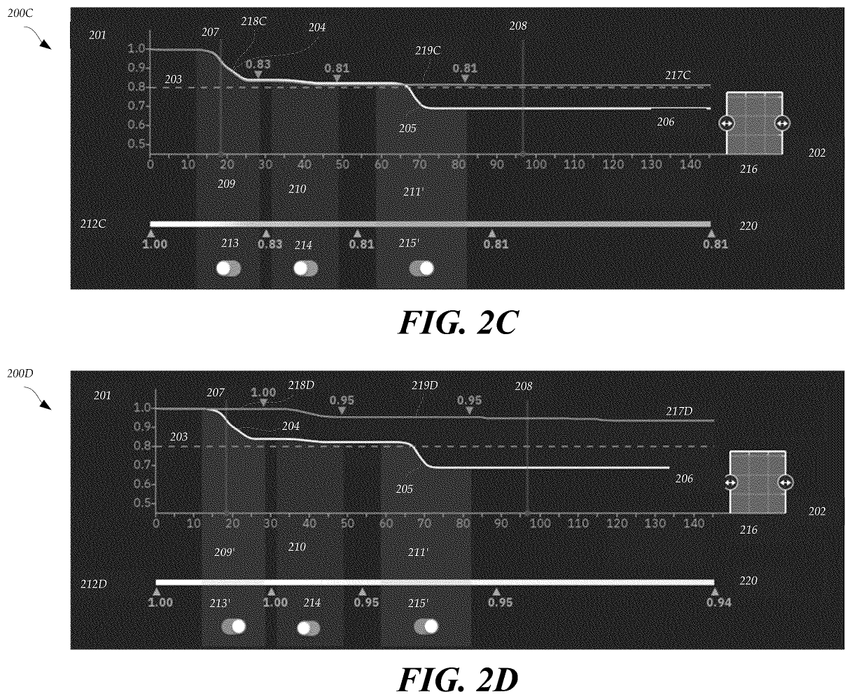

The adjustable lesion graph described herein, for example, with respect to FIGS. 2A-2D, may therefore succinctly allow for the medical professional to analyze a patient's cardiac health options. For example, prior software-based tools may incorrectly indicate that nulling a particular lesion will be associated with a greatest index gain. In this example, the tools may identify the particular lesion having the greatest index gain. However, due to crosstalk effects it may be more advantageous to simulate a healthy vessel area at a different lesion, or a set of lesions.

This application therefore improves upon prior software-based analysis techniques. Additionally, the adjustable lesion graph provides for a holistic view into a patient's current health while flexibly detailing health options. In this way, the medical professional may more accurately determine which lesion of a set of lesions to simulate. The user interface cohesion simplifies such determinations and may additionally provide for a helpful aid to a patient in understanding his/her options.

In some embodiments, a system or user device may generate the adjustable lesion graph based on cardiac images of a patient's heart. For example, the cardiac images may represent angiographic images and may be obtained through minimally invasive procedures, such as a catheter inserted into the artery, or non-invasive procedures, such as a computer tomography (CT) scan. The cardiac images may be obtained, for example, from an imaging system (e.g., a c-arm) that captures the heart from different perspectives or viewpoints. The system or user device may then, in some embodiments, generate a three-dimensional model of the patient's heart or a portion thereof. The three-dimensional model may indicate diameters associated with vessels included in the patient's heart. In this way, the system or user device may identify lesions, compute an indices indicative of vascular function, and so on, based on the three-dimensional model. Additional description related to the above is included in U.S. Pat. No. 10,595,807, which is hereby incorporated herein by reference in its entirety.

In some embodiments, additionally or alternatively, the techniques described herein may be applied to vasculature of another organ, for example, a kidney, a retina, and/or a brain. It should be understood, where cardiac vasculature is described in particular, that implicit reference is also made to embodiments relating to the vasculature of another organ, with changes as necessary as would be clear to one skilled in the art.

### Block Diagram-Lesion Impact System

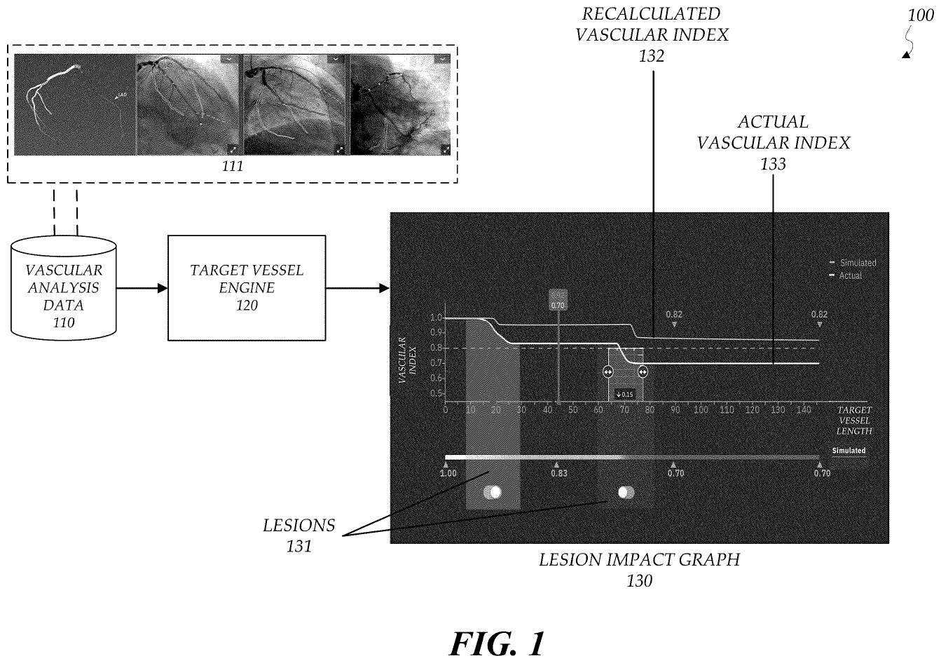

FIG. 1 is a block diagram of an example lesion impact system 100 generating an adjustable lesion graph 130 of a targeted vessel. As described herein, the adjustable lesion graph 130 may include information identifying lesions along a vessel, or vessels, with the lesions' associated index drops. In some embodiments, the lesion impact system 100 can include lesions in the adjustable lesion graph 130 if the lesion is associated with an index drop greater than a threshold, for example, to illustrate the lesions with the highest impact. In some embodiments, the medical professional can determine what the index drop threshold is. In some embodiments, the index drop threshold can be minimal or such that any index drop is considered a lesion.

The lesion impact system 100 may represent a system of one or more processors, such as a computer, a tablet, a mobile device, a wearable device, and so on. The system 100 may execute an application which generates a coronary physiology assessment 111, which can include the adjustable lesion graph 130 described herein. The coronary physiology assessment 111, which can be referred to as a cardiac model, can include data that was derived from angiographic images, such as, but not limited to, a 3D cardiac model(s) (3D model), the geometry information of the vessel (such as vessel width and length), lesion locations based on the geometry information, 2D cardiac model(s), the index values or index drops at locations along one or more vessels, and so on. The system 100 may additionally present user interfaces which include information determined from an outside system (e.g., a cloud or server system). In some instances, the user interfaces can allow the user to adjust the data displayed in the coronary physiology assessment 111 and/or generate additional data related to the patient's coronary vasculature.

The lesion impact system 100 may determine the information which forms the adjustable lesion graph 130, which may be collectively referred to as a cardiac model, based on cardiac images, such as angiographic images. The angiographic images can be stored in a vascular analysis database 110. The system 100 may analyze the angiographic images to identify one or more target vessels in the target vessel engine 120. A three-dimensional model may then be created, and estimations of vessel diameter may be effectuated along the length of the vessels. These vessel diameters may be used, for example, to inform the identification of lesions along the vessels. Thus, the system 100 may determine, or access, information identifying positions of lesions and diameters across the length of the vessel.

The system 100 may additionally determine indices indicative of vascular function along the length of the vessels. As an example, the system 100 may determine index drops, such as reductions in fractional flow reserve (FFR) value, downstream in the vessel. Example index drops may be attributed to stenoses or lesions for which medical intervention may be advantageous. As illustrated, the graph 130 may map or plot the vascular index (e.g., FFR value) or index drops (e.g., from a starting or normalized value, such as 1) based on length along the vessel. For example, individual locations along the length of vessel may have individual vascular indices or index drops.

Additional disclosure related to determining a three-dimensional model, an index indicative of vascular function, and so on, are described in more detail in U.S. Pat. No. 10,595,807, which is hereby incorporated herein by reference in its entirety.

In the illustrated example, the adjustable lesion graph 130 depicts the left anterior descending (LAD) artery or vessel. Thus, the adjustable lesion graph 130 includes information related to narrowing of the LAD along its length. In FIG. 1, the length extends from 0 to a threshold length which may represent a unit of measurement associated with the length of the LAD. In the example, the graph 130 includes the length along a first axis (e.g., the x-axis).

Along a second axis, the adjustable lesion graph 130 includes values for an index indicative of vascular function (referred to in FIG. 1 as a vascular index). For example, the index may represent a fractional flow reserve (FFR) value which may have an upper limit of 1.0. As known by those skilled in the art, lesions may cause a reduction in FFR which is referred to herein as an index drop.

Thus, the adjustable lesion graph 130 maps index values to position along the length of the LAD. A line, referred to as the actual vascular index 133, represents a patient's index as it changes along the length of the LAD. As illustrated, there are two lesions 131 with one lesion being positioned between about 20 and 70 units along the first axis. The first lesion is associated with an index drop to above about 0.8 and second is associated with an index drop to about 0.7 with about a 0.15 index drop change. In some embodiments, an index value below 0.8 may be considered an abnormal value (e.g., index drops or pressure changes associated with the effects of CAD).

Toggles are positioned proximate to positions of the lesions 131 (e.g., user interface elements which allow for toggling). As mentioned above, a user may provide user input to the adjustable lesion graph 130 to toggle on or off a lesion. As described herein, the user may null a lesion by toggling the nulling effect of the lesion on. An update to the adjustable lesion graph 130 based on a user toggling a lesion on can include determining an “unstenosed,” or non-stenotic, state of a blood vessel segment, and in particular, an estimate of the geometry of a blood vessel as if a region of stenosis were opened therein, modeling the effect on a healthy blood vessel segment.

Nulling the lesion therefore causes the effect of the lesion to be removed. For example, in some embodiments the system 100 may update the diameter associated with the lesion to be open (e.g., normal or that which is associated with an index drop less than a threshold). In this example, the system 100 can determine updated index values based on the updated diameter for the length of the vessel associated with the lesion (e.g., 20 and 70 units as an example).

Determining index values is described in more detail with respect to at least U.S. Pat. No. 10,595,807. Since the index values are determined using the diameters or radii of the lesions, and updated or nulled lesions, the effects of crosstalk between lesions can be addressed. In some instances, the diameters of the lesions can be determined from the 3D vascular tree model.

The lesion impact system 100 can indicate to a user the vascular index 133 and the lesions 131 along the target vessel and allow user input to null out a selected lesion to generate an at least partially healthier vascular system than the originally generated coronary physiology assessment 111. The adjustable lesion graph 130 can update to indicate to the user the simulated vascular index 132 along the length of the target vessel without the effect of selected nulled lesions. In some instances, to null out a selected lesion can be by increasing the diameter of the lesioned area to a diameter considered normal and/or healthy. A normal and/or healthy diameter can be when there is no drop in the index values at the lesioned area.

In some embodiments, a nulled lesion can be directly implemented into a 3D vascular tree model generated in the coronary physiology assessment 111 for assessment. For example, the nulled lesion can include modifications of vascular width beyond the length of the area of the lesion, reflecting, for example, the elasticity of the vascular wall. In some embodiments, the nulled lesion is simulated in 2-D by forming a deformation in a vascular wall's 2-D image that simulates a healthy vessel diameter, then re-importing the modified image into the model. This method can preserve information about the implantation site's curvature and surrounding environment. Furthermore, updating 2-D images taken from more than one angle can result in a complete assessment of the implant environment. Moreover, adjustable lesion graph 130 can apply more than one nulled lesion to determine the impact of nulling more than one lesion.

In some embodiments, lesion nulling can include identifying portions of the vasculature relatively unaffected by disease. In such embodiments, the disease can be, for example, stenotic (narrow) regions of the vasculature, or in others, for example, can be aneurysm (widened) regions of the vasculature. Construction of the healthy vasculature, in some embodiments, can estimate a healthy vascular lumen size based on the healthiest vascular regions nearby which can be identified. Additionally or alternatively, identification of a healthy region comprises the substitution of a homologous vessel—for example, a vessel from another portion of the same vascular tree or a portion of another vascular tree that is matched to the modeled vascular tree in terms of one or more parameters such as age, gender, and/or body size.

In some embodiments, healthy vasculature simulation can include using later- and/or earlier-obtained images of a vascular tree. In some embodiments, the earlier images are used, in whole or in part, as a non-stenotic baseline or as a basis for determining a non-stenotic state. In such embodiments, the non-stenotic vascular state is extrapolated, based, for example, on the determination that certain regions can be observed to close over time, reflecting their stenotic character.

In other embodiments, healthy vasculature simulation comprises the propagation of a healthy vessel region width estimation into a region of width change due to disease. The propagation comprises, for example, extrapolation between non-diseased vascular regions across regions that are or may be diseased.

In some embodiments, propagation comprises weighting the presumptively most healthy areas to be more important in setting a vascular width than other regions. Healthy vascular regions may be, for example, relatively wide regions and/or those nearest to a simulated value based on location in the vasculature and/or other parameters such as patient vital statistics.

### Example Adjustable Lesion Graphs

FIGS. 2A-D illustrates example user interfaces which include an example adjustable lesion graph 130 with different nulling effects of lesions toggled on, or off, by a user. The user interfaces may be examples of user interfaces rendered by an application executing on a system or user device. The user interfaces may also represent front-end user interfaces associated with a web application.

FIGS. 2A-D illustrates the same example adjustable lesion graph 200A-D of a targeted vessel with different combinations of nulled lesions toggled on. The example adjustable lesion graphs 200A-D of the targeted vessel illustrates three different lesions along the length of the target vessel whose nulling can be toggled on to update the vascular index.

FIG. 2A illustrates an example information of an adjustable lesion graph 200A with three lesions along the length of the target vessel and no lesion nulling toggled on to update the vascular index analysis of the target vessel. The adjustable lesion graph 200A depicts an actual vascular index graph 206 of the vascular index 201, such as FFR, along the target vessel length 202. The target vessel length 202 can be a measurement along the target vessel selected. In some embodiments, the target vessel length 202 can be in millimeters.

As depicted in the adjustable lesion graph 200A, at approximately 68 mm, the actual vascular index graph 206 crosses the vascular index stenosis threshold 203, set at 0.8. As disclosed above, an FFR value below 0.8 can indicate the potential presence of stenosis. Therefore, as indicated by the actual vascular index graph 206 being under the vascular index stenosis threshold 203 from approximately 68 mm to 140 mm, this portion of the vessel is determined to have potential to indicate the presence of stenosis in the overall vessel due to the lesions' physiological impacts. As indicated in the adjustable lesion graph 200A, there are three lesions along the target vessel length 202, a first lesion 209 at approximately 15-29 mm, a second lesion 210 at approximately 31-49 mm, and a third lesion 211 at approximately 59-82 mm.

The lesion impact system 100 can determine these lesions by determining a vascular state score for each, such as a SYNTAX value, and comparing it to a lesion threshold. Additional description related to the above is included in U.S. Pat. No. 10,943,233, which is hereby incorporated herein by reference in its entirety. In some embodiments, for vessel narrowing that does not meet the index drop threshold to be considered a lesion, the adjustable lesion graph 200A includes a user-selected lesion 216. The user-selected lesion 216 can provide a user with an option to indicate a portion of the vessel, which did not meet an index drop threshold, to be analyzed or to implement a nulled lesion. In some instances, the user-selected lesion 216 can be represented by an adjustable box that can be re-positioned by the user along the length of the vessel. Similar to the lesion impact system 100 determining a boundary for each of the lesions meeting the index drop threshold, the user can adjust the size of the user-selected lesion 216 box to increase or decrease the boundary of the user-selected lesion. For further insight, a first lesion actual impact 204 can be shown to result in a 0.16 point drop as a result of the first lesion 209, and a third lesion actual impact 205 can be shown to result in a 0.13 point drop as a result of the third lesion 211.

Although the adjustable lesion graph 200A does not implement them, the adjustable lesion graph 200A depicts a first lesion toggle 213, a second lesion 214, and a third lesion toggle 215 that can implement a nulled lesion for a lesion toggled on. In some instances, the analysis or nulling of the user-selected lesion 216 can be automatic in response to the user-selected lesion 216 box being dragged over a portion of the vessel length or in response to a user indication. In some instances, the user indication for the user-selected lesion 216 can be implemented as a toggle similar to the other lesion toggles 213, 214, 215.

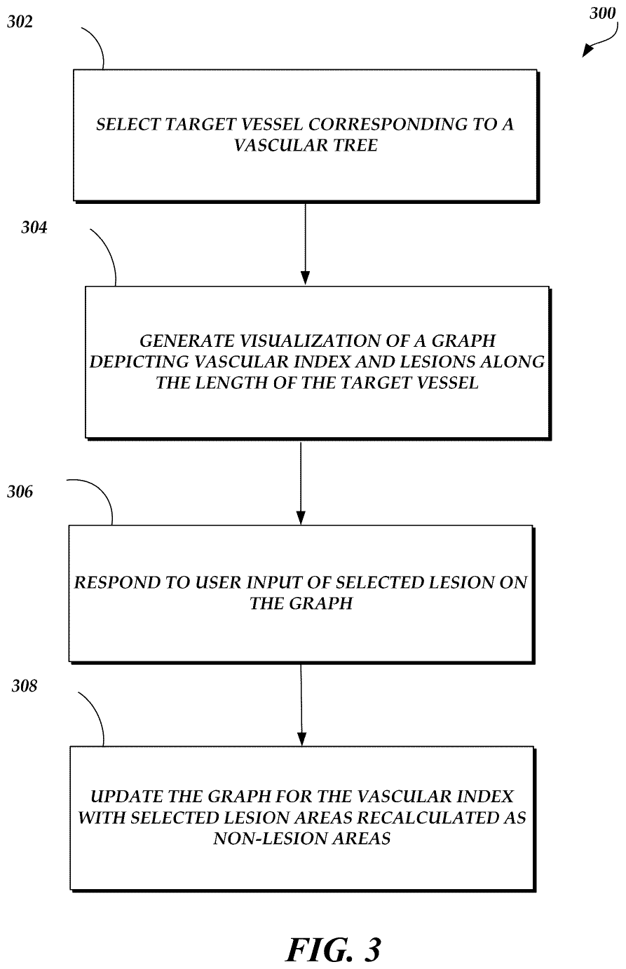

An adjustable lesion graph 200A can also include a vascular index color graph 212A that depicts the vascular index along the target vessel length 202. As illustrated, the graph includes a starting index value of 1 and an ending of 0.69. In some embodiments, as lesions are nulled the ending index value may be updated in real time (e.g., as described in block 308 of FIG. 3, this ending index value may be presented as a single index value for presentation to the user). In some embodiments, the vascular index color graph 212 can be color coated so that different vascular index values correspond to different colors. For example, a vascular index value of 1.00 can be represented by white, and a vascular index color of 0.50 or less can be represented by black.

In some embodiments, the values between 1.00 and 0.50 can gradually shift between colors. For example, the values of 1.00 to 0.9 can gradually shift from white to yellow, the values of 0.9 to 0.75 can gradually shift from yellow to orange, the values of 0.75 to 0.65 can gradually shift from orange to red, and the values of 0.65 to 0.5 can gradually shift from red to black. In alternative embodiments, the values between 1.00 and 0.50 can be a grayscale shifting from white and darkening until it reaches black. The colors presented are not intended to be limiting, as the system can use any color for any vascular index value and does not require a gradual shift.

In some embodiments, the vascular index color graph 212A can depict numerical representations of the vascular index 201 along the target vessel length 202. In some embodiments, the location of the vascular index color graph 212 can mirror the x-axis's target vessel length 202 so that the vascular index illustrated on the vascular index color graph 212 corresponds to the position along the target vessel length 202. In some embodiments, the numerical representations can be at points of interest, including the beginning and end of the target vessel and any points near a lesion. Additionally, the adjustable lesion graph 200A can include a user-selected actual vascular index 208 to select a point along the target vessel length 202 to indicate the vascular index 201 at said point. Similar to the vascular index color graph 212A, the actual vascular index 208 markers can correspond to the color associated with the vascular index 201 at the marked point.

In some embodiments, the first lesion actual impact 204, the second lesion actual impact, third lesion actual impact 205, and similar impact scores may not be depicted. In some embodiments, a maximal 3D stenosis diameter 207 regarding the target vessel diameter drop can be depicted. The maximal 3D stenosis diameter 207 can be the region of the actual vascular index graph 206 with the highest gradient. This highest gradient can correspond to the area along the target vessel with the drop in the index value.

FIG. 2B illustrates example information of an adjustable lesion graph 200B with three lesions along the length of the target vessel with a first lesion toggled on 213′ to update the vascular index analysis of the target vessel to show the impact of the nulled first lesion 209′.

The adjustable lesion graph 200B in FIG. 2B is the same as the adjustable lesion graph 200A in FIG. 2A, except as noted. The adjustable lesion graph 200B depicts a first lesion toggled on 213′ that implements a nulled first lesion 209′. This results in a simulated vascular index graph 217B of the vascular index 201 along the target vessel length 202, without the effect of the first lesion. The simulated vascular index graph 217B can be compared to the actual vascular index graph 206 to visualize changes in index drops at areas of the lesion, such as a first lesion simulated impact 218B compared to the first lesion actual impact 204 and a third lesion simulated impact 219B compared to the third lesion actual impact 205. Even if a particular lesion is not nulled, the simulated index drop can differ from the actual index drop as a result of cross-talk effects from the nulled lesions. As depicted by the simulated vascular index graph 217B, a nulled lesion at the first lesion provides a higher FFR value in the first half of the target vessel but still results in the potential presence of stenosis with the remaining lesions.

The value beyond 70 mm is below the vascular index stenosis threshold 203. The shift in drop can be illustrated by a first lesion simulated impact 218B compared to first lesion actual impact 204, which depicts a huge increase. However, the drop at the third lesion is still significant, as depicted by the simulated vascular index graph 217B at a third lesion simulated impact 219B dropping relatively equivalent to the third lesion actual impact 205.

In some instances, the portion of the vessel length covered by the user-selected lesion 216 can be associated with a sectional change of the index along the portion of the vessel length for the actual vascular index graph 206 and the simulated vascular index graph 217B. The sectional change can be a difference, change, or measure of central tendency, of a value determined by the user along the portion of the vessel that the user dragged the user-selected lesion over, from the beginning of the user-selected lesion 216 box to the end of the user-selected lesion 216 box. The value determined by the user can be a change of index, a change of vessel diameter, or other values represented in the coronary physiology assessment 111. For example, if the user-selected lesion 216 was dragged to cover the portion of the vessel from 10 units to 30 units, not shown, then a sectional change of the index for the actual vascular index graph 206 can be displayed as 0.18 to represent the drop of the index from about 1.0 at 10 units to just above 0.8 at 30 units, along with a section change of the index for the simulated vascular index graph 217B displayed as 0.00 to represent the lack of drop of the index from about 1.0 at 10 units maintained as 1.0 at 30 units.

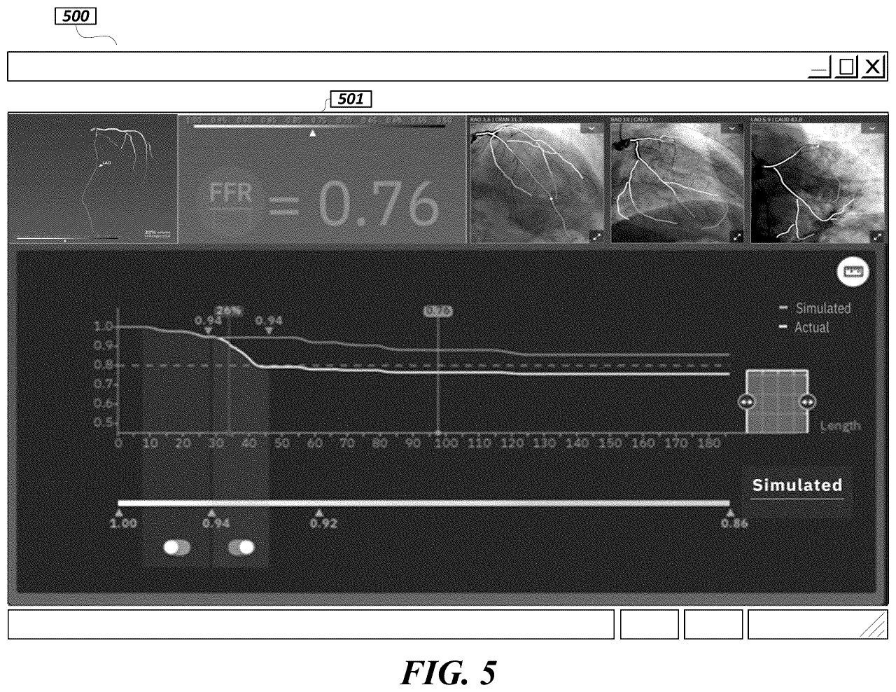

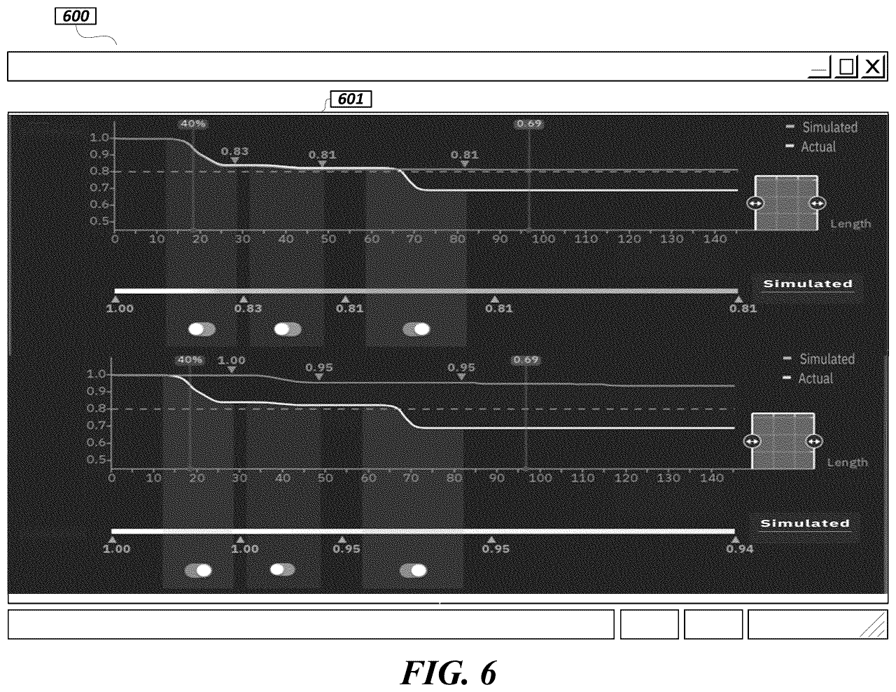

The 200B also includes an updated vascular index color graph 212B corresponding to the simulated vascular index graph 217B. Additionally, when a lesion nulling is toggled, the lesion impact system 100 may denote that the vascular index color graph 212 has “updated values” as shown in FIGS. 5-6.

FIG. 2C illustrates example information of an adjustable lesion graph 200C with three lesions along the length of the target vessel with a third lesion toggled on 215′ to update the vascular index analysis of the target vessel to show the impact of the nulled third lesion 211′. The adjustable lesion graph 200C in FIG. 2C is similar to the adjustable lesion graph 200B in FIG. 2B but is analyzed using a nulled lesion on the third lesion 211 instead of the first lesion 209. As such, the values of the simulated vascular index graph 217C, first lesion simulated impact 218C, and updated vascular index color graph 212C are adjusted accordingly. As indicated by the simulated vascular index graph 217C, the nulled third lesion 211′ indicates a low potential presence of stenosis, as the simulated vascular index graph 217C remains above 0.8 throughout target vessel length 202. Thus, even though the first lesion 209 caused a 0.3 larger drop in FFR than the third lesion 211, a nulled third lesion 211′ can be more effective than a nulled first lesion 209′ at indicating a lack of potential presence for stenosis.

FIG. 2D illustrates example information of an adjustable lesion graph 200D with three lesions along the length of the target vessel and the first lesion toggled on 213′ and the third lesion toggled on 215′ to update the vascular index analysis of the target vessel to show the impact of the nulled first lesion 209′ and the nulled third lesion 211′. The adjustable lesion graph 200D in FIG. 2D is similar to the adjustable lesion graph 200B-C in FIG. 2B-C but is analyzed using a nulled lesion on both the first lesion 209 and third lesion 211, rather than a single respective lesion. As such, the values of the simulated vascular index graph 217D, first lesion simulated impact 218D, first lesion simulated impact 218D, and updated vascular index color graph 212D are adjusted accordingly. As illustrated in the adjustable lesion graph 200D, the simulated vascular index graph 217D maintains a high FFR due to the nulled first lesion 209′ and nulled third lesion 211′.

### Example Flowchart

FIG. 3 is a flowchart of an example process for generating an adjustable lesion graph 130, 200A-D that can be updated to provide information on a lesion's impact on the vascular index of a target vessel. For convenience, process 300 will be described as being performed by a system of one or more computers (e.g., the lesion impact system 100).

At block 302, the system selects a target vessel corresponding to a vascular tree. The vascular tree can be part of the cardiac model, which can include either a 3D model of the cardiac vasculature or a 2D angiographic image. As described above, the vascular tree can be obtained from vascular analysis data store 110 storing a cardiac model, such as from a previously completed coronary physiology assessment 111 or one obtained in real-time during a catheterization procedure.

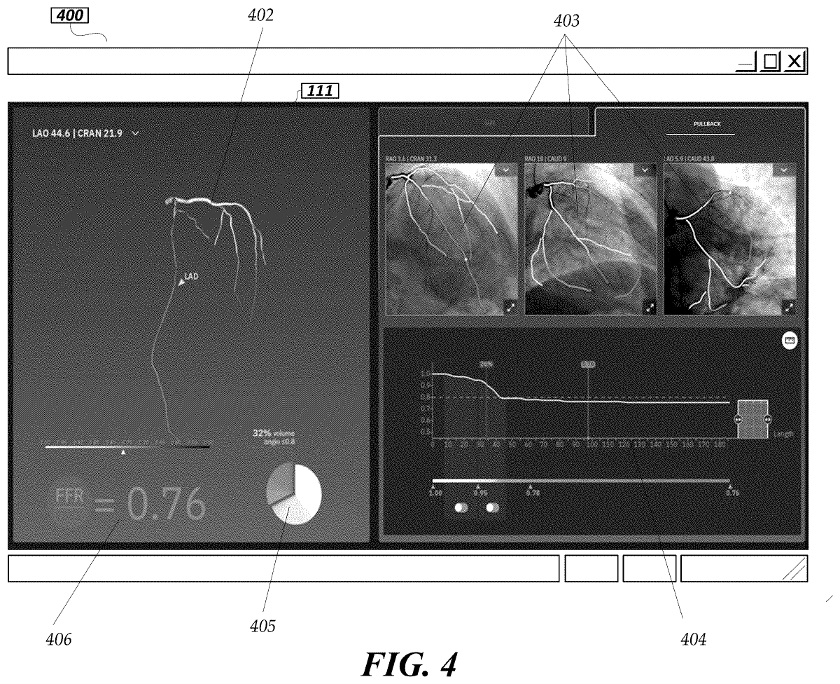

At block 304, the system generates a visualization of a graph depicting the vascular index and lesions along the length of the target vessel. The system can already have the graph generated in the vascular analysis data store 110 as part of the coronary physiology assessment 111, as show in FIG. 4 depicting an adjustable lesion graph 404. As described above, determining a lesion can relate to a vascular state scoring, such as a SYNTAX score.

At block 306, the system responds to the user input of selected lesions on the graph. As described above, a user may toggle a button corresponding to a particular lesion to analyze the vessel by nulling the lesion. In some embodiments, a user can select portions of the vessel that are identified as lesions. The system can then null the selected lesions and reanalyze the vascular index along the target vessel.

At block 308, the system updates the graph for the vascular index with the selected lesion areas recalculated as non-lesion areas. As described above, the system may overlay a simulated vascular index graph 217B-D over an actual vascular index 208, as well as other alterations described above, to indicate a physician about the simulated results of implementing a stent for a particular lesion. In some embodiments, the system may present an index value which represents a final index value at a threshold distance from a distal end of the target vessel (e.g., which includes the lesions). For example, it may be similar or the same value as the ending index value in element 212A. As an example, when nulling the lesion the index value after the lesions may be increased due to the removal of the effects of the lesion. Thus in some embodiments this single index value may be presented such that the user can cycle through toggling on/off lesions and view the corresponding index value.

### User Interface

FIGS. 4-6 illustrate example user interfaces identifying aspects of the features described herein. These user interfaces may be rendered partially by a computer system implanting the lesion impact system 100 or its alternative embodiments.

FIG. 4 is a user interface 400 illustrating a vascular assessment 401. As illustrated, the vascular assessment 401 can include an actual vascular index 406 at a target lesion, an adjustable lesion graph 404, a vascular index pie chart 405 depicting the overall health of the target vessel by illustrating what volume of the target vessel has an index below a threshold value and a visual indicator on how the index values are distributed on the target vessel, a 3D model 402 of the cardiac vasculature, and the 2D angiographic images 403 with a color coating. The adjustable lesion graph 404 illustrated in FIG. 4 is similar to the adjustable lesion graph 200A in FIG. 2A can include information similar to 200A but only depicts two lesions instead of three. The vascular index along the length of the target vessel has a subtle drop at the first lesion and a more significant drop at the second lesion, as illustrated, which may indicate ischemia.

FIG. 5 is a user interface 500 illustrating an alternative embodiment of a coronary physiology assessment 111 with an exploded view of the adjustable lesion graph. The adjustable lesion graph illustrated in FIG. 5 depicts the same target vessel as the adjustable lesion graph 404 in FIG. 4, but with the second lesion nulling toggled on. As illustrated, the adjustable lesion graph is similar to the adjustable lesion graphs 200B-C in FIGS. 2B-D, as they can include the simulated vascular index graph generated by toggling the second lesion on and the updated color graph indicator. As illustrated, nulling out the second lesion can provide an estimate on the impact of said lesion on the potential presence of CAD. In some embodiments, the adjustable lesion graph may not display a simulated lesion simulated impact, as illustrated.

FIG. 6 is a user interface 600 illustrating a comparison between the adjustable lesion graph 200C in FIG. 2C and the adjustable lesion graph 200D in FIG. 2D to provide a user with a visualization of the impact of the first lesion on the atherosclerosis if the third lesion is assumed to be healthy. As described above, although nulling out just the third lesion still illustrates the potential presence of CAD, nulling out both the first and third lesions can illustrate meaningly impact on the potential presence of CAD.

### Other Embodiments

All of the processes described herein may be embodied in, and fully automated, via software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence or can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and engines described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, the term “about” refers to within +10%.

The terms “comprises”, “comprising”, “includes”, “including”, “having”, “such as” and their conjugates mean: “including but not limited to”.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, and medical arts.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It should be emphasized that many variations and modifications may be made to the above-described embodiments, the elements of which are to be understood as being among other acceptable examples. All such modifications and variations are intended to be included herein within the scope of this disclosure.

1. A method implemented by a system of one or more processors, the method comprising:
accessing a cardiac model of a portion of a patient's heart, the portion including one or more vessels of the patient's heart, and the cardiac model indicating a plurality of lesions along a length of at least one of the vessels;
obtaining, based on the cardiac model for the lesions, respective positions along the length for which the lesions are associated with index drops, wherein the index drops are with respect to an index indicative of vascular function; and
causing presentation of a user interface, wherein the user interface:
presents a graph mapping individual positions along the length of the vessel and values of the index indicative of vascular function,
presents individual user interface elements enabling nulling of individual lesions, and
updates the graph in response to received user input to one or more of the user interface elements, wherein the user input nulls effects of one or more lesions.

2. The method of claim 1, wherein the user interface elements are toggles.

3. The method of claim 1, wherein the plurality of lesions comprises at least one user-identified lesion.

4. The method of claim 1, wherein the plurality of lesions further comprises at least one automatically-identified lesion that meets an index drop threshold.

5. The method of claim 1, wherein the cardiac model includes a three-dimensional model generated based on angiographic images.

6. The method of claim 1, wherein the index indicative of vascular function is a fractional flow reserve value.

7. The method of claim 1, wherein the graph is a line.

8. The method of claim 1, wherein the user input triggers a determination of index drops associated with remaining lesions.

9. The method of claim 1, wherein nulling a lesion cause updating of diameters of the vessel which are associated with the lesion, and wherein the updated diameters are updated to cause index drops less than a threshold.

10. The method of claim 1, wherein the user interface updates to include an updated index indicative of vascular function.

11. (canceled)

12. (canceled)

13. (canceled)

14. (canceled)

15. The method of claim 1, wherein the index drops are with respect to reductions in fractional flow reserve value, and wherein updating the index indicative of vascular function includes updating the index drops along the length of the at least one of the vessels.

16. The method of claim 1, wherein the updating the index indicative of vascular function includes identifying a reduction in index a threshold distance from a distal end of the at least one vessel.

17. (canceled)

18. (canceled)

19. A system comprising one or more processors and non-transitory computer storage media storing instructions that when executed by the one or more processors, cause the one or more processors to perform operations comprising:
accessing a cardiac model of a portion of a patient's heart, the portion including one or more vessels of the patient's heart, and the cardiac model indicating a plurality of lesions along a length of at least one of the vessels;
obtaining, based on the cardiac model for the lesions, respective positions along the length for which the lesions are associated with index drops, wherein the index drops are with respect to an index indicative of vascular function for the vessels; and
based on information indicating that a particular lesion of the plurality of lesions is to be nulled, updating the index indicative of vascular function to null effects of the particular lesion.

20. The system of claim 19, wherein updating the index is based on addressing crosstalk associated with remaining lesions.

21. The system of claim 19, wherein the particular lesion is downstream from one or more remaining lesions.

22. The system of claim 19, wherein the particular lesion is between adjacent of the lesions.

23. The system of claim 19, wherein the index drops are with respect to reductions in fractional flow reserve value, and wherein updating the index indicative of vascular function includes updating the index drops along the length of the at least one of the vessels.

24. The system of claim 19, wherein the updating the index indicative of vascular function includes identifying a reduction in index a threshold distance from a distal end of the at least one vessel.

25. (canceled)

26. (canceled)

27. Non-transitory computer storage media storing instructions that when executed by a system of one or more computers, cause the one or more computers to:
access a cardiac model of a portion of a patient's heart, the portion including one or more vessels of the patient's heart, and the cardiac model indicating a plurality of lesions along a length of at least one of the vessels;
obtain, based on the cardiac model for the lesions, respective positions along the length for which the lesions are associated with index drops, wherein the index drops are with respect to an index indicative of vascular function; and
cause presentation of a user interface, wherein the user interface:
presents a graph mapping individual positions along the length of the vessel and values of the index indicative of vascular function,
presents individual user interface elements enabling nulling of individual lesions, and
updates the graph in response to received user input to one or more of the user interface elements, wherein the user input nulls effects of one or more lesions.

28. The computer storage media of claim 27, wherein the index drops are with respect to reductions in fractional flow reserve value, and wherein updating the index indicative of vascular function includes updating the index drops along the length of the at least one of the vessels.
